(19) United States
(12) Patent Application Publication
(10) Pub. No.: US 2006/0134157 A1
(43) Pub. Date: Jun. 22, 2006

(54) Co-continuous phase composite polymer blends for in-vivo and in-vitro biomedical applications

(75) Inventors: Richard Lehman, Princeton, NJ (US); James Idol, Columus, OH (US); Thomas Nosker, Stockton, NJ (US); Richard Renfree, Scotch Plains, NJ (US); Jennifer Lynch, Franklin Park, NJ (US); Kenneth Van Ness, Lexington, VA (US)

Correspondence Address: SYNNESTVEDT LECHNER & WOODBRIDGE LLP, P O BOX 592, PRINCETON, NJ, 08542-0592, US

(73) Assignee: RUTGERS, THE State University, New Brunswick, NJ (US)

(21) Appl. No.: 10/516,964

(22) PCT Filed: Jun. 6, 2003
(86) PCT No.: PCT/US03/18107

§ 371 Date: Jul. 27, 2005

Related U.S. Application Data
(60) Provisional application No. 60/385,883, filed on Jun. 6, 2002.

(51) Int. Cl. B29C 67/20; B32B 3/06; B32B 3/26; B32B 5/22; A61F 13/00

(52) U.S. Cl. 424/422; 264/41; 428/304.4; 428/306.6; 428/315.5; 428/317.9

(57) ABSTRACT

Tissue-compatible polymer composites characterized by a co-continuous, integrated multi-phase, three-dimensional microstructured network of two or more immiscible biocompatible polymers.

### CROSS REFERENCE TO RELATED APPLICATION

The present application claims priority benefit under 35 U.S.C. §119(e) of U.S. Provisional Patent Application Ser. No. 60/385,883 filed Jun. 6, 2002, the disclosure of which is incorporated by reference.

### BACKGROUND OF THE INVENTION

Materials used for human implants and tissue growth scaffolding require several key properties that include sufficient strength and toughness, compatibility with tissue environments, biochemical durability, avoiding release of moieties that stimulate body rejection mechanisms, and proper surface characteristics to promote adhesion of adjacent tissue. The compatibility of the implant or scaffolding with specific types of tissue is particularly important with regard to induction of tissue growth and conductive growth regimes to produce viable tissue development in-vivo.

This direction has led to the evaluation of numerous types of porous scaffolding structures made from inorganic biocompatible materials such as hydroxyapatite and various polymers. The functionality of hydroxyapatite type materials is principally derived from the biochemical similarity of this material with the inorganic phase of bone tissue. The functionality of the porous polymer materials fall into three distinct categories: [1] materials that are passive in vivo, [2] materials that degrade in vivo and produce benign or growth promoting degradation products, and [3] materials that can be doped with drugs and other biochemical agents that promote growth, reduce inflammation or generate other desirable tissue characteristics in-vivo.

However, mechanical and biochemical compatibilization of the implant structure with the body environment continues to be a key issue in implant development. Overall the ideal scaffold or implant material is one that, as a bulk material, is fundamentally passive to the body chemistry but which provides a mechanical surface texture that promotes tissue induction and conduction over an appropriate, tailorable period of time that corresponds to tissue healing dynamics. Furthermore, the mechanical properties of the scaffold/tissue assembly should remain constant during the healing/transition process to allow sufficient member loading to further stimulate hard tissue growth.

Bone scaffolding materials, for example, need the ability to be engineered to give tailored solubility properties. On the one hand, the development of interpenetrating porosity networks are essential to provide osteoinductive growth channels necessary for the development of natural tissue. On the other hand, dissolution of the substrate cannot proceed so quickly that in-vivo scaffold modulus is lost too early, thus jeopardizing the process and the structure before sufficient natural tissue can form.

Composites with integrated, interpenetrating networks formed from immiscible polymer blends are described in U.S. Pat. No. 5,298,214, hereby incorporated by reference. This patent discloses that polystyrene can be blended with a “mixed plastics” component from a recycling stream to produce materials that behave mechanically and appear morphologically like fiber reinforced composites. Moreover, there exists a unique composition range within which mechanical properties are optimized. In this range, the morphology, as determined by scanning electron microscopy, of both the polystyrene component and predominantly polyolefin component, obtained from the “mixed plastics,” exist as a dual phase micro-structure with both components forming three dimensional networks that are integrated and interpenetrating with one another. See also Renfree et al., “Dual Phase, Co-Continuous Morphology from Mixtures of Recycled Polystyrene/Curb-side Tailings Materials,” SPE ANTEC '92, pp. 2396-2400, hereby incorporated by reference.

In this type of dual phase co-continuous structure, two phases intertwine in such a way that both phases remain continuous throughout the material. The morphology is analogous to that of a sponge soaked in water where both sponge and water form continuous systems. Determining composition ranges at which dual phase co-continuity occurs can be estimated by selecting the volume ratio of the two blend components to approximately equal the viscosity ratio.

Based on experimental observations that the phase with the lower viscosity or the higher volume fraction, tended to form the continuous phase, Jordhamo et al, Polym. Eng. Sci., 26(8), 517 (1986), suggested a semi-empirical expression which relates the region of expected dual phase co-continuity to the viscosity ratio and volume ratio of the blend components. Their paper asserts that the condition of dual phase co-continuity can be achieved by the application of shear to a polymer blend system close to the phase inversion region. As described by Equation (1), the model predicts that phase inversion should occur when the viscosity ratio and the volume ratio are about equal, i.e., when

$$\frac{{V}_{A}}{{V}_{B}} \approx \frac{{η}_{A}}{{η}_{B}} \qquad ( 1 )$$

wherein ηᵢ is the viscosity of phase i and Vᵢ is the volume fraction of phase i. As can be seen, the model sets the viscosity ratio as being approximately equal to the volumetric ratio. The material described in U.S. Pat. No. 5,298,214 exhibits this two-phase microstructure. One phase consists essentially of polystyrene and the other consists essentially of polyolefin.

Unfortunately, biomedical implants cannot be made from waste plastic recycling streams. Furthermore, while the co-continuous polymer phases of U.S. Pat. No. 5,298,214 form three-dimensional integrated interpenetrating networks, there is no disclosure regarding how either phase can at least in part be removed or otherwise replaced with a three dimensional interpenetrating network of pores to form a structure suitable for biomedical implantation.

### SUMMARY OF THE INVENTION

It has now been discovered that immiscible tissue-compatible polymer combinations will form co-continuous, composite multi-phase, three-dimensional integrated interpenetrating micro-structure networks when blended and formed according to the process described by U.S. Pat. No. 5,298,214, and, furthermore, because such polymers can be selected to erode at different rates, the more rapidly eroding polymer(s) will dissolve first and leave behind a three-dimensional interpenetrating microstructure network of pores that promote tissue ingrowth. Thus, polymers already considered acceptable for the fabrication of biocompatible tissue implants can be formed into tissue implants in such a way that one or more phases can be absorbed by adjoining tissues to form a three-dimensional interpenetrating porous microstructure that promotes the ingrowth of adjoining tissue into the implant.

Therefore, according to one aspect of the present invention, a tissue-compatible polymer composite is provided having a co-continuous, integrated multi-phase, three-dimensional micro-structured network of two or more immiscible biocompatible polymers. The polymer composites of the present invention exhibit desirable mechanical properties. Therefore, it is not necessary for any of the polymer components of the composite to bioerode or dissolve, particularly if tissue ingrowth is unlikely and long term mechanical properties must be maintained.

However, the ability to select one or more polymer components to dissolve faster than one or more of the others to form three-dimensional interpenetrating porous microstructures that promote tissue ingrowth is particularly advantageous. Therefore according to one embodiment of this aspect of the invention, at least one polymer component of the composite is bioerodible, and erodes at a rate faster than at least one other polymer component of the composite.

Three-dimensional interpenetrating microstructured porous networks can be formed in the composites of the present invention having at least one bioerodible polymer either in vivo or in vitro. That is, the composite with at least one bioerodible polymer can be pore-free prior to implantation, so that the interpenetrating microstructured porous networks form as the bioerodible polymer dissolves and is absorbed by adjoining tissues to permit ingrowth of the very same tissue into the pores that form. Alternately, the one or more bioerodible polymers can be dissolved and removed as part of the manufacturing process to provide a tissue compatible polymer implant with an interpenetrating microstructured porous network. The dissolving of an erodible polymer under such circumstances is essentially conventional, with the results depicted in FIGS. 2-6. For example, the polymer can be removed from the composite structure by contacting the composite with aqueous solutions of the type employed for in vitro testing of polymer bioerodibility under conditions essentially similar to in vitro testing. Alternately, at least one polymer can be removed by contact with a solvent for the polymer.

Therefore, according to another aspect of the present invention, a porous tissue-compatible polymer structure is provided having a three-dimensional microstructured porous network. When the polymer structure begins with more than two polymers, and more than one remain after one or more are removed, the polymer portion of the structure is a co-continuous, integrated multi-phase, three-dimensional microstructured network of two or more immiscible biocompatible polymers.

Porous composites according to this aspect of the present invention may have at least one polymer phase completely or partially removed in vitro to create a full or partial network of pores for tissue ingrowth. There is no lower limit on the amount of polymer phase removed because even the slightest removal of polymer will create a composite with a textured surface that promotes tissue adhesion.

Thus, according to another aspect of the present invention, a method is provided for forming porous tissue compatible polymer structures having three-dimensional microstructured porous networks, including the steps of providing a tissue-compatible polymer composite having a co-continuous, integrated multi-phase, three-dimensional microstructured network of two or more immiscible biocompatible polymers, at least one of which is bioerodible, and dissolving in vitro at least a portion of a bioerodible polymer.

The polymer composites of the present invention, with or without porous networks formed in vitro, can be fabricated into medical implant devices by essentially conventional means. Therefore, another aspect of the present invention provides biocompatible medical implant devices formed from the polymers of the present invention. Medical implant device include porous polymer scaffolds for tissue engineering and tissue-guided regeneration applications.

The co-continuous polymer blend technology allows for the incorporation of substances into one or more of the polymers at the blend interface to promote bone or tissue growth, such as cell attachment mediators, osteoinductive substances, cellular growth factors, other nutrients and pharmaceuticals, and the like. Particulate materials that promote bone or tissue growth may also be used, such as hydroxyapatite or tricalcium phosphate. Therefore, tissue-compatible polymer composites according to the present invention will also include composites containing one or more substances or particles that promote bone or tissue ingrowth, nutrient substances, pharmaceutical substances, and the like.

The polymer composites of the present invention can be used as cell growth substrates, either in vivo or in vitro. Therefore, still yet another aspect of the present invention includes a method of regulating cellular attachment, migration and proliferation on a polymeric substrate, characterized by contacting living cells, tissues or biological fluids containing living cells with the polymer composites of the invention.

The foregoing and other objects, features and advantages of the present invention are more readily apparent from the detailed description of the preferred embodiments set forth below taken in conjunction with the accompanying drawings.

### BRIEF DESCRIPTION OF THE DRAWINGS

FIG. 1 depicts the range of co-continuous regions for an A-B polymer mixture;

FIG. 2 depicts a 64/36 wt % etched PMMA/PLA composite material of the present invention at 350× viewed parallel to the extrusion axis;

FIG. 3 depicts the etched composite of FIG. 2 at 700× viewed parallel to the extrusion axis;

FIG. 4 depicts the etched composite of FIG. 2 at 1000× viewed parallel to the extrusion axis;

FIG. 5 depicts the etched composite of FIG. 2 at 300× viewed perpendicular to the extrusion axis;

FIG. 6 depicts the etched composite of FIG. 2 at 1300× viewed perpendicular to the extrusion axis;

FIG. 7 depicts the results of a PBS aging study for a PMMA/PLA composite material of the present invention;

FIG. 8 depicts the results of a PBS aging study for another PMMA/PLA composite material of the present invention;

FIG. 9 the results of a deionized water aging study for the PMMA/PLA composite material of FIG. 8; and

FIG. 10 depicts a comparison of modulus values averaged over time for polymer composites of the present invention and unblended polymers.

### DETAILED DESCRIPTION OF THE PREFERRED EMBODIMENTS

The tissue compatible composites are prepared using the co-continuous polymer blend technology disclosed by U.S. Pat. No. 5,298,214. Prior experience with other polymer systems, particularly the polystyrene/high density polyethylene system, has revealed that co-continuous composites can be produced from immiscible polymers by melt processing. The key feature needed to achieve these composites is proper composition percentages plus high shear melt processing, such as that encountered with proper screw configuration and machine operation in melt extrusion and injection molding. The application of the technology to immiscible tissue compatible polymers is essentially conventional. The invention resides in the recognition that such polymers can be processed using this technology to form biocompatible composite materials, and the unique and unexpected properties resulting possessed by the composite materials.

The polymer composites are based on well-known tissue compatible polymers. Essentially any biocompatible polymer that is thermally and otherwise stable under the processing conditions of the polymer blend technology is suitable for use with the present invention. Examples of suitable polymers include α-hydroxycarboxylic acids and copolymers thereof, including PGA, PLA and copolymers thereof; the polyethylene oxide/polyethylene terephthalate disclosed by Reed et al., Trans. Am. Soc. Artif. Intern. Organs, page 109 (1977); and the copolymers of lactic or glycolic acid or combinations of the two with hydroxy-ended flexible chains, preferably poly(alkylene glycols) of various molecular weights, disclosed by U.S. Pat. No. 4,826,945. Other suitable polymers include biodegradable and biocompatible polycaprolactones, polyhydroxybutyrates and copolymers of polyesters, polycarbonates, poly-anhydrides and poly(ortho esters), poly(alkylene oxides) such as poly(ethylene glycol) (PEG), polysaccharides, poly(vinyl alcohol), polypyrrolidone, poly(acrylic acid), poly(ethylene-co-vinyl acetate), (commonly referred to by its abbreviation EVA), poly(ortho-esters), poly-vinylpyrrolidone, pyran copolymer, poly(hydroxypropyl-methacrylamide-phenol), poly (hydroxyethyl-aspartamide-phenol), poly(ethylene oxide)-poly(lysine) substituted with palmitoyl residues, poly(hydroxybutyric acid), polyacetals, poly(dihydropyran), poly(cyano-acrylates), polyarylates, polyurethanes and poly(alkylene oxide ethers), and cross-linked and amphipathic block copolymers of hydrogels, and the like.

Bisphenol-A based polyphosphoesters have also been suggested for use in biodegradable scaffold design. Such polymers include poly(bisphenol-A phenylphosphate), poly(bisphenol-A ethylphosphate), poly(bisphenol-A ethylphosphonate), poly(bisphenol-A phenylphosphonate), poly[bis(2-ethoxy)hydrophosphonic terephthalate], and copolymers of bisphenol-A based poly(phosphoesters). Although these polymers have been suggested in U.S. Pat. No. 5,686,091, the known cytotoxicity of bisphenol-A make them less preferred candidates for implantation. On the other hand, another useful polymer system is the copolymers of poly-ethylene oxide/polyethylene terephthalate.

Polymers within each polymer family have varying rates of degradation depending upon polymer structure, molecular weight and other well-understood factors. Thus, one of two polymers within a family may be the faster dissolving polymer component of a composite according to the present invention, while other of the two polymers may be the slower dissolving component in a different composite according to the present invention.

The composite is formed by blending two or more immiscible polymers. The term immiscible, synonymous with non-miscible, is used in its ordinary sense with respect to the polymers as defined by Billmeyer, Textbook of Polymer Science (3rd Ed., John Wyley & Sons, 1984). One of ordinary skill in the art can easily select two or more immiscible polymers for processing without undue experimentation. For example low water solubility polymers, or water-insoluble polymers can be used as the phase that dissolves more slowly or not at all, and water soluble polymers can be employed for phases that are intended to dissolve. Combinations of water soluble polymers with polymers that are water insoluble or have lower water solubility are generally good candidates because these materials will usually be non-miscible.

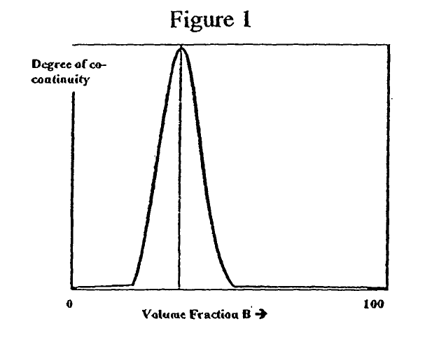

The proper composition ratio of two (or more) constituent polymers is defined by the simple ratio given in Equation (1) that requires only rheological characterization of the polymers over the thermal processing range of the extruder or injection molder. Eliminating V_{B} by the relationship V_{A}+V_{B}=1, it becomes directly apparent that if the viscosity ratio is 0.5, for example, the volume fraction of the more fluid component should be one-third. A range of co-continuous regions exists and is centered on or near the predicted composition as shown qualitatively in FIG. 1 for an A-B polymer mixture. A wide range of polymer molecular weights can be used to obtain a range of performance. Component ratios will vary depending upon the viscosity and volume fraction for the molecular weights of the polymers selected. The component weight ratios of co-continuous regions will typically range between about 15:85 and about 85:15, preferably between about 25:75 and about 75:25, and more preferably between about 30:70 and about 70:30%.

Porous polymer composites can be prepared in vitro, as discussed above, by removing at least one polymer phase from a polymer composite of the present invention. In addition to promoting tissue ingrowth, porosity also increases the surface area of the biorerodible polymer when it is in contact with the organic fluids of the body, thereby increasing the rate of bioerosion. Alternately, porosity may be introduced by foaming one or more polymer phases during the composite-forming process, or adding a particulate material to one or more polymer phases, such a salt or a sugar, or introducing the particulate material at the polymer phase interface, and then removing the particulate material with a solvent therefor, such as water.

Foaming can be performed in a variety of ways, the simplest of which for polymers such as poly(lactic acid) is to keep the polymer in a controlled nonzero humidity environment prior to blending in the extruder. Other methods include compounding in foaming agents like azodicarbonamide or others. Putting a particulate material such as a sugar at the interface of the immiscible polymers can be performed by simply dry mixing the immiscible polymers and the sugar prior to extrusion.

Preferred polymer combinations include a blend of poly(methyl methacrylate) (PMMA) and either poly(lactic acid) (PLA), poly(glycolic acid) (PGA), copolymers thereof or two or more thereof. Co-continuous mixtures are generated that provides unique opportunities for achieving the desirable traits of a hard tissue implant/scaffold material. A wide variety of PLA and PGA polymers and copolymers can be used to obtain a range of desirable biomaterial properties and the current invention is not limited to any one composition.

The PMMA/PLA and/or PGA ratio can be varied considerably to take advantage of various molecular weight polymers and to alter the texture of the two-phase system. Processing and fabrication technologies enable structures to be formed with special properties. This is applicable to essentially any combination of polymers forming a composite of the present invention.

For example, with composites containing PGA, PLA, or copolymers or mixtures thereof, a given implant application may require a high level of lactic acid nutrient at the outset, but less as the growth process becomes established. Extruding or injection molded implant structures may be prepared that have graded profiles of PLA and/or /PGA and PMMA. The microstructure of the PLA and/or PGA/PMMA blend is tailored to meet a variety of end-use biomedical requirements.

Blends will contain both PMMA and PLA, PGA and/or copolymers thereof as co-continuous phases. The PMMA will be the structural phase that provides the necessary strength to the structure and PLA, PGA, and/or copolymers thereof provide a slowly soluble biodegradable phase that produces an evolving osteoinductive/conductive morphology. In addition to engineering the biochemical environment as the PLA and/or PGA dissolves to nutritionalize the surrounding tissues to stimulate ingrowth, the two-phase structure imparts increased toughness to the implant structure and the porosity generated by the dissolution of the PLA and/or PGA phase promotes adhesion sites for adjacent bone, muscle, or ligament tissue.

One of the special features of this polymer system is the similarity of solubility parameters for PMMA and PLA and PGA polymers and copolymers. Although actual values vary and depend on specific molecular weights and compositions, the solubility parameters are usually on the verge of immiscibility/miscibility. This borderline immiscibility and the processing of these two polymers in a manner that generates a co-continuous distribution of both polymers is a key feature of this embodiment of the present invention and enables several key properties. The near miscibility of the two phases enables the formation of much stronger interfacial bonds that would be possible in fully immiscible systems. Furthermore, the processing of these polymers into a co-continuous distribution maximizes the interfacial surface area, enhances the interaction between the two polymers, and enables a continuous inductive/conductive tissue growth channel to develop when the PLA, PGA and/or copolymer thereof is removed by biochemical action. In addition, if a material is desired that has minimal or no biodegradation, i.e. where tissue growth is unlikely and long term mechanical properties must be retained, the PMMA and PLA/PGA polymers can be processed to produce a miscible alloy that inhibits or greatly reduces selective degradation and channel formation.

The use of PMMA in polymer composites is also advantageous because it promotes the fastening of the composite to bone or other tissues with super glue-type adhesives better than almost any other implant material, because such adhesives are based on PMMA and related polyacrylates.

The co-continuous polymer blend technology also allows the possibility of incorporating substances into one or more of the polymers at the blend interface to promote bone or tissue growth, such as hydroxyapatite or tricalcium phosphate. These particulates, as well as other nutrients and pharmaceuticals, can also be combined in the bioerodible phase (such as the PLA or PGA) phase to provide the conditions necessary for robust tissue growth and adhesion. Examples of pharmaceutical substances include cell attachment mediators, biologically active ligands, and substances that enhance or exclude particular varieties of cellular or tissue ingrowth. Such substances include, for example, osteoinductive substances, such as bone morphogenic proteins (3 MP), epidermal growth factor (EGF), fibroblast growth factor (FGF), platelet-derived growth factor (PDGF), insulin-like growth factor (IGF-I and II), TGF-β and the like. Other suitable pharmaceutical substances include antibiotics and blood clotting inhibitors.

Pharmaceutical substances are added in quantities effective to provide dosage levels between about 0.001 mg/kg to about 1000 mg/kg, preferably from about 0.01 mg/kg to about 100 mg/kg, and more preferably from about 0.10 mg/kg to about 20 mg/kg. For each pharmaceutical substance, individual determinations may be made to determine the optimal dosage required. The determination of effective dosage levels, that is, the dosage levels necessary to achieve the desired result, will be within the ambit of one skilled in the art. The release rate of the pharmaceutical sunbstances may also be varied within the routine skill in the art to determine an advantageous profile.

Particulate substances, such as hydroxyapatite and tricalcium phosphate, as well as nutrient substances, are employed in larger quantities, typically between about 0.5 and about 20 vol. %, preferably between about 1 and about 15 vol. %, and more preferably between about 2 and about 10 vol %.

The substances can be incorporated into one or more polymer phase for subsequent release in a controlled fashion by conventional drug delivery means. The substances may be released by a bioerosion of the polymer phase, or by diffusion from the polymer phase. Alternatively, the substance may migrate to the polymer surface, where it is active, or the substance may be covalently attached to a polymer.

The polymer composites are shaped into articles for tissue engineering and tissue guided regeneration applications, including reconstructive surgery. The evolving porous structure allows generous cellular ingrowth, eliminating the need for cellular preseeding. The polymer composites may also be molded to form external scaffolding for the support of in vitro culturing of specialized cells and tissues for the creation of external support organs. The scaffold functions to mimic the extracellular matrices (ECM) of the body. The scaffold serves as both a physical support and an adhesive substrate for isolated cells during in vitro culture and subsequent implantation. As the transplanted cell populations grow and the cells function normally, they begin to secrete their own ECM support. The scaffold polymer is selected to degrade as the need for an artificial support diminishes.

In the reconstruction of structural tissues like cartilage and bone, tissue shape is integral to function, requiring the molding of the scaffold into articles of varying thickness and shape. Any crevices, apertures or refinements desired in the three-dimensional structure can be created by removing portions of the composite with scissors, a scalpel, a laser beam or any other cutting instrument. A fabrication sequence may be employed that involves producing large quantities of polymer composite to meet a specific end use, wherein the final shape of the implant or other component is determined by tomography and is stored in a CAD/CAM image file. The image file is then sent to a CNC (computer numerical controlled) milling machine that produces a net shape part to exact specifications

Scaffold applications include the regeneration of tissues such as nervous, musculoskeletal, cartilaginous, tendenous, hepatic, pancreatic, ocular, integumenary, arteriovenous, urinary or any other tissue forming solid or hollow organs.

The scaffold may also be used in transplantation as a matrix for dissociated cells such as chondrocytes or hepatocytes to create a three-dimensional tissue or organ. Any type of cell can be added to the scaffold for culturing and possible implantation, including cells of the muscular and skeletal systems, such as chondrocytes, fibroblasts, muscle cells and osteocytes, parenchymal cells such as hepatocytes, pancreatic cells (including Islet cells), cells of intestinal origin, and other cells such as nerve cells and skin cells, either as obtained from donors, from established cell culture lines, or even before or after genetic engineering, and embryonic and non-embryonic stem cells. Pieces of tissue can also be used, which may provide a number of different cell types in the same structure.

The cells are obtained from a suitable donor, or the patient into which they are to be implanted, dissociated using standard techniques and seeded onto and into the scaffold. In vitro culturing optionally may be performed prior to implantation. Alternatively, the scaffold is implanted, allowed to vascularize, then cells are injected into the scaffold. Methods and reagents for culturing cells in vitro and implantation of a tissue scaffold are known to those skilled in the art.

The following non-limiting examples set forth hereinbelow illustrate certain aspects of the invention. All parts and percentages are by weight unless otherwise noted and all temperatures are in degrees Celsius.

### EXAMPLES

A series of composite materials were prepared and evaluated as described below.

Materials. Polymethylmethacrylate (PMMA) was obtained from GE corporation in the form of pellets suitable for extrusion processing. Two grades of polylactic acid (L210 and L207S) were obtained from Boehringer Ingelheim Corporation, Germany in the form of granular powders. Both materials are pure lactides with molecular weights in the range of 113,000 to 300,000 as shown in Table 1. Approximate physical properties for both materials are given in Table 2.

TABLE 1
Molecular Weight Calculations for PLA

| | | | Inherent | | | | |
|---|---|---|---|---|---|---|---|
| | Boehringer | | Viscosity | Mark- | Mark- | Mᵥ = | |
| Trade Name | Material No. | Lot Number | (dl/g) | Houwing K | Houwing a | ([η]/K)^{1/n} | [η]= KMᵥ^{a} |
| PLA L210 | 60640645 | 10005490 | 3.9 | 1.29E − 04 | 0.82 | 291068 | 3.90E + 00 |
| PLA L207S | 51923 | 1005010 | 1.8 | 1.29E − 04 | 0.82 | 113368 | 1.80E + 00 |

Note: Inherent Viscosity from Boerhinger Specification Sheets; value for L210 is the average value [3.4,4.4]. Constants K and a from Boehringer Spec: Fischer, Stenzel, Wegner, Kolloid-Z. and Z. Pol. 251.980 (1973). Approximation used: Intrinsic Viscosity=Inherent Viscosity

TABLE 2
Approximate Physical Properties for PLA 2075 and PLA 210

| Property | Value |
|---|---|
| Tensile strength at 37°, 50 mm/min [MPa] | 82.5 |
| Strain at yield at 37°, 50 mm/min [%] | 3.6 |
| Young's Modulus, E, at 37° [MPa] | 670 |
| Flexural Strength, at 37°, 50 mm/min [MPa] | 118 |
| Notched Impact Strength at 37°, [J] | 0.41 |
| MFI [g/10 min] | 2.7 |
| Drying temperature | 140 |
| Minimum drying time, [h] | 4 |
| Ideal drying time, [h] | 8 |
| Glass transition, Tg | 57 |
| Crystallization Temperature, TX | 180 |
| Mark-Houwing Constants | K = 1.29 × 10⁻⁴ |
| | a. = 0.82 |
| Density at 22° [g/cm 3] | 1.256 |

For the PLA(L207S)/PMMA composites, the viscosity of the PMMA at 200° is 3989 and the viscosity of the PLA is 1563 Pa's, yielding a volume fraction of PLA of 28.2% as the center of the co-continuous region. Similarly, for the PLA(L210)/PMMA composites, the viscosity of the PMMA at 200° is 3989 and the viscosity of the PLA is 3739 Pa's, yielding a volume fraction of PLA of 48.4% as the center of the co-continuous region. Thus, a broad areas of the PMMA/PLA composition space is related to the current invention. Since small amounts of PLA in a largely PMMA matrix are of limited interest, the most relevant range of composition space in this system is from 15% PLA to 85% PLA, more preferably from 25 to 70% PLA, and mostpreferably from 30-60% PLA by volume.

Two representative compositions that were processed as described in this section are shown in Table 3.

TABLE 3
Viscosity Values and Composite Proportions for
Two PLA/PMMA Composites

| | PMMA | PLA | Vis- | Vol. | Vol. | | |
|---|---|---|---|---|---|---|---|
| PLA | Viscosity | Visc. | cosity | Fraction | Fraction | Wt. % | Wt. % |
| Type | (Pa's) | (Pa's) | Ratio | PMMA | PLA | PMMA | PLA |
| L210 | 3989 | 3739 | 1.067 | 0.516 | 0.484 | 50.2 | 49.8 |
| L207S | 3989 | 1563 | 2.552 | 0.718 | 0.282 | 70.7 | 29.3 |

These compositions were processed in a Brabender single screw laboratory extruder. The extruder screw was 0.75″ in outside diameter, had a root diameter of 0.655″ and was fitted to a barrel with inside diameter of 0.755″. The screw motor was set at 100 RPM, which generated a shear rate of 75 sec 1 and the polymer was heated to 200°. No die was used, the formulated polymer composite was extruded from the barrel opening which produced rods of approximately 10 mm diameter that were cut into convenient lengths. These lengths, when cooled were milled into rods and disks for subsequent testing in various solutions. Samples were fractured in liquid nitrogen to provide clean fracture surfaces for the SEM image work. In addition to a range of PMMA/PLA compositions, this work demonstrated that hydroxyapatite particles could be successfully blended with the molten polymers in the extruder. Approximately 2.7% by weight of micron sized hydroxyapatite particles were added to were added to polymer mixtures, blended, and processed in the extruder.

Results

Materials. The extruded polymer composites were of good quality and texture. The appearance of the materials varied from nearly clear and transparent to substantially foggy and translucent, apparently an indication of crystallinity and immiscibility.

Calorimetry and Dynamic Mechanical Analysis (DMA). Scanning Calorimetry and DMA were conducted to assess the degree of crystallinity and immiscibility. Some samples contained substantial crystallinity, as expected from the PLA whereas others showed only a faint thermal signal at the crystallization temperature. Similarly, the 57° Tg of PLA and the 104° Tg of PMMA were seen in some samples, whereas other samples showed an intermediate Tg, indicating some degree of alloying. It is this mixed behavior of this special system that enables a wide range of biological performance to be engineered by simply altering the composition and molecular weight of the PLA and by altering the composite composition and processing conditions.

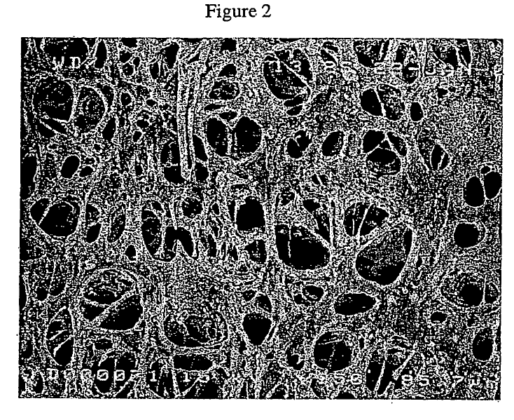

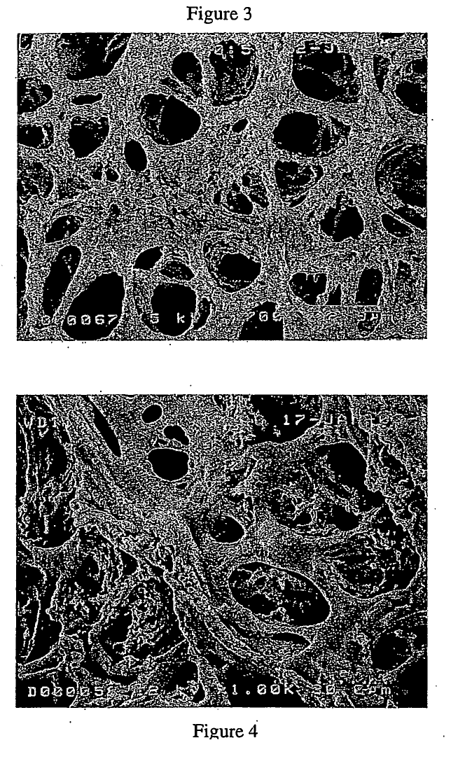

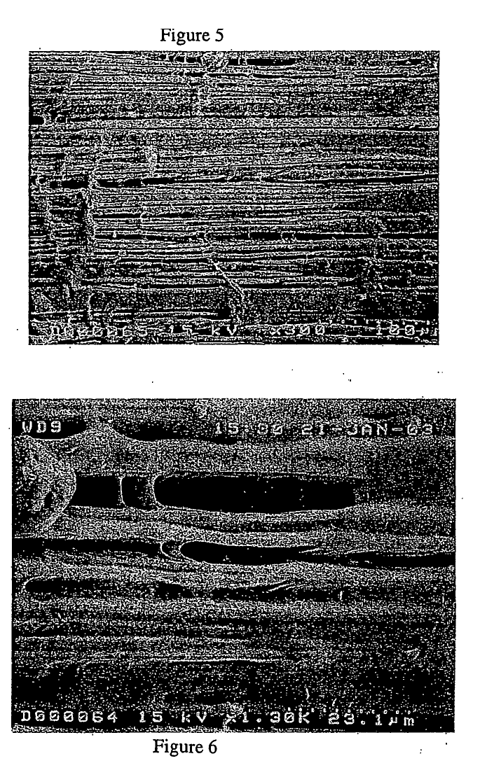

Structure. One of the most striking features of these materials is the apparent two-phase structure that is observed in compositions processed in the co-continuous range. As shown in the photomicrographs (FIGS. 2-6) of a 64/36% PMMA/PLA composite that was etched (dimethyl formate, 15 seconds, 25° C.) to reveal the microstructure, the resulting materials are clearly co-continuous. The degradable phase, PLA, has been removed and the remaining PMMA phase clearly illustrates the desirable conductive channels. FIGS. 2-4 illustrate the morphology as viewed parallel to the extrusion axis and FIG. 5-6 illustrate the fibrous/channel structure as viewed perpendicular to the extrusion axis.

Properties

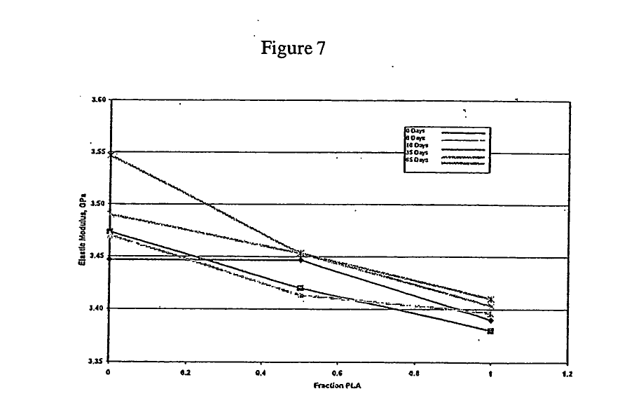

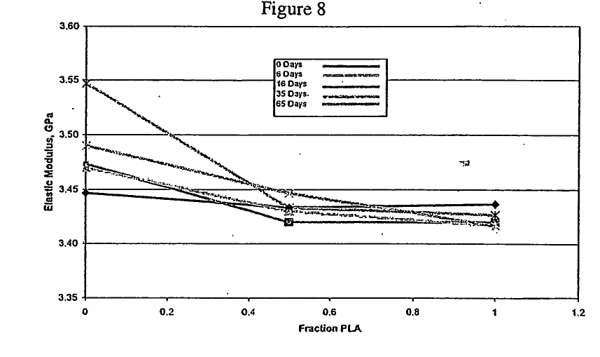

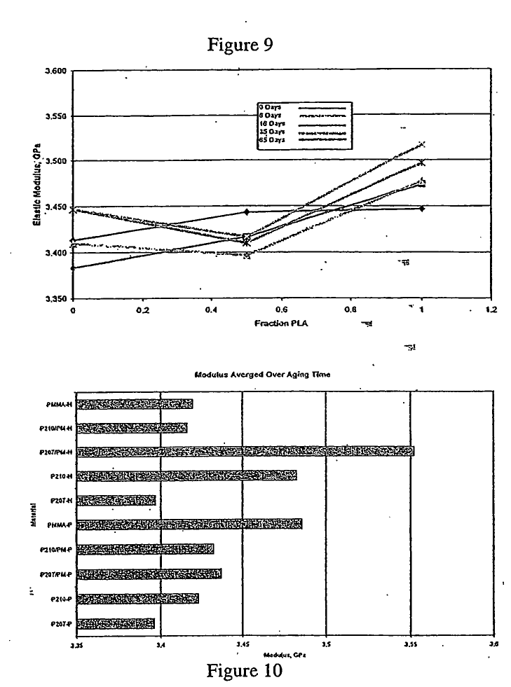

Phosphate buffer solutions aging. Small bars were cut from some of the materials produced so that they could be aged at 37° in a phosphate buffer solution that provides an approximation of in vivo conditions. The goal of this testing was to demonstrate the ability of PLA-PMMA co-continuous composites to retain modulus over a two month period. The solubility of PLA in vivo is well known and is the basis for its current use at dissolvable sutures, among other uses. By combining it with PMMA in a co-continuous structure, the PLA tissue compatibility properties can be employed while at the same time slowing the rate at which modulus is lost due to dissolution. FIGS. 7-10 show good retention of modulus over the 65-day test period.

The present invention thus provides highly biosensitive structures that simulate in-vivo conditions for promoting cellular growth and tissue repair. The foregoing examples and description of the preferred embodiment should be taken as illustrating, rather than as limiting, the present invention as defined by the claims. As would readily be appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

What is claimed is:

1. A tissue-compatible polymer composite comprising a co-continuous, integrated multi-phase, three-dimensional microstructured network of two or more immiscible biocompatible polymers.

2. The polymer composite of claim 1, wherein, at least one polymer component of the composite is bioerodible, and erodes at a rate faster than at least one other polymer component of the composite.

3. The polymer composite of claim 1, consisting essentially of first and second polymer components.

4. The polymer composite of claim 3, wherein said second polymer component is bioerodible.

5. The polymer composite of claim 4, wherein said first polymer component is bioerodible and erodes at a rate slower than said second polymer component.

6. The polymer composite of claim 3, wherein the weight ratio of the first component to the second component is between about 15:85 and 85:15.

7. The polymer composite of claim 4, wherein said first polymer component is PMMA and said second polymer component is selected from the group consisting of PLA, PGA and copolymers thereof.

8. The polymer composite of claim 2, comprising PMMA and at least one polymer selected from the group consisting of PLA, PGA and copolymers thereof.

9. The polymer composite of claim 1, wherein at least one polymer phase comprises one or more substances or particles that promote bone or tissue ingrowth.

10. The polymer composite of claim 9, wherein at least one polymer phase comprises particles of hydroxyapatite or tricalcium phosphate.

11. The polymer composite of claim 1, wherein at least one polymer phase comprises one or more nutrient or pharmaceutical substances.

12. The polymer composite of claim 1, wherein at least one polymer phase is foamed.

13. A porous tissue-compatible polymer structure comprising a three-dimensional microstructured porous network.

14. The porous polymer structure of claim 13, wherein the polymer portion of the structure is a co-continuous, integrated multi-phase, three-dimensional microstructured network of two or more immiscible biocompatible polymers.

15. The porous polymer structure of claim 14, wherein said polymer portion comprises first and second polymer components, wherein said second polymer component is bioerodible.

16. The porous polymer structure of claim 15, wherein said first polymer component is bioerodible and erodes at a rate slower than said second polymer component.

17. The porous polymer structure of claim 15, wherein said first polymer component is PMMA and said second polymer component is selected from the group consisting of PLA, PGA and copolymers thereof.

18. The porous polymer structure of claim 13, wherein the polymer component of said structure comprises one or more substances or particles that promote bone or tissue ingrowth.

19. The porous polymer structure of claim 18, wherein said polymer component comprises particles of hydroxyapatite or tricalcium phosphate.

20. The porous polymer structure of claim 13, wherein the polymer component of said structure comprises one or more nutrient or pharmaceutical substances.

21. An implantable medical device or tissue scaffold formed from the composite of claim 1, 2, 3, 4, 7, 8, 9, 10 or 11.

22. A method of forming porous tissue compatible polymer structures having three-dimensional microstructured porous networks, comprising the steps of providing a tissue-compatible polymer composite having a co-continuous, integrated multi-phase, three-dimensional microstructured network of two or more immiscible biocompatible polymers, and dissolving in vitro at least a portion of at least one polymer.

23. A method of regulating cellular attachment, migration and proliferation on a polymeric substrate, comprising contacting living cells, tissues or biological fluids containing living cells with the polymer composites of claim 1.
